(19) United States
(12) Patent Application Publication
(10) Pub. No.: US 2007/0141089 A1
(43) Pub. Date: Jun. 21, 2007

(54) Topical composition comprising terbinaf ine adn hydrocortisone

(76) Inventors: Catherine Larnier, Founex (CH); Michel Steiger, La-Tour-de-Peliz (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/566,874

(22) PCT Filed: Aug. 11, 2004
(86) PCT No.: PCT/EP04/08994

§ 371 Date: Jun. 29, 2006

(30) Foreign Application Priority Data
Aug. 12, 2003 (US) 03018271.1

(51) Int. Cl. A61K 31/573; A61K 31/137; A61K 9/00

(52) U.S. Cl. 424/400; 514/171; 514/649

(57) ABSTRACT

The invention relates to topical pharmaceutical compositions comprising terbinafine and hydrocortisone. Said compositions exhibit beneficial antimycotic properties, especially against dermatophytes.

The invention relates to topical pharmaceutical compositions with antimycotic activity, more specifically anti-dermatophyte activity and anti-yeast activity.

Dermatophytes are fungi that can cause infections of the skin, hair and nails due to their ability to utilize keratin. The organisms colonize the keratin tissues and cause fungal infections, e.g. known as tinea or ringworm, in association with the infected body part. The organisms are transmitted by either direct contact with infected host (human or animal) or by direct or indirect contact with infected exfoliated skin or hair In combs, hair brushes, clothing, furniture, theatre seats, caps, bed linens, towels, hotel rugs and locker room floors. Depending on the species the organism may be viable in the environment for up to 15 months. There is an increased susceptibility to infection when there is a pre-existing Injury to the skin such as scares, burns, marching, excessive temperature and humidity.

Candida species, in contrast to dermatophytes, are yeasts. They are normally present in humans and usually become pathogenic only in case of overgrowth, often induced by local factors like immunodepression. Yeasts like Candida are opportunistic agents and usually need co-factors to become pathogenic, predominantly systemically.

The topical application of terbinafine in the treatment of fungal infections, such as mycoses, especially dermatomycoses caused by dermatophytes, e.g. athlete's foot (=tinea pedis), jock itch (=tinea cruris), ringworm or onychomycosis, and dermatomycoses caused by yeasts, e.g. sweat rashes or seborrheic dermatitis, is known in the art.

It has now surprisingly been found that by topical application of terbinafine together with hydrocortisone the antimycotic properties are improved In an unexpected manner. This is particularly so because topical hydrocortisone is known to be a corticosteroid of low potency. Surprisingly, the combination of the present invention is particularly beneficial in fighting dermatophytes. As already outlined above, the latter are the main cause for superficial mycoses frequently occurring in humans, such athlete's foot, jock itch or ringworm. Treatment of said superficial mycoses is generally Improved, and a faster healing achieved, by use of the specific combination of the invention. This is quite surprising in view of the fact that terbinafine is known to be rather effective in the eradication and treatment of dermatophytes even when applied alone. In this respect, it has surprisingly been found that the addition of hydrocortisone does not negatively interfere with the antimycotic activity of terbinafine.

With the combination of the present invention, the cure of superficial mycoses, e.g. athlete's foot, is in general achieved more quickly and a quicker relief of typical symptoms, such as itching, erythema, vesiculation, burning or fissures, is observed.

Therefore, the invention relates to a pharmaceutical composition adapted to topical administration comprising terbinafine, or a topically acceptable salt thereof, and hydrocortisone, or a topically acceptable ester or salt thereof, together with at least one topically acceptable carrier.

Terbinafine is known and e.g. described in The Merck Index, Twelfth Edition, 1996, under No. 9299. It is commercially available under the trademark LAMISIL. Topically acceptable salts thereof are e.g. terbinafine hydrochloride, terbinafine lactate or terbinafine ascorbate. Preferred are terbinafine (=free base) and terbinafine hydrochloride.

In the topical compositions of the invention, terbinafine is typically present In an amount of from 0.1 up to 10%, especially of from 0.2 up to 5%, and In particular of from 0.5 up to 2%, of the total composition on a weight basis. All percentages given in this document are weight-% (w/w), if not indicated otherwise.

Hydrocortisone is known and e.g. described in The Merck Index, Twelfth Edition, 1996, under No. 4828. Topically acceptable esters and salts thereof are e.g. hydrocortisone acetate, e.g. the 21-acetate; hydrocortisone butyrate, e.g. the 17-butyrate; hydrocortisone valerate, e.g. the 17-valerate; hydrocortisone 21-phosphate disodium salt or hydrocortisone 21-sodium succinate. Preferred are hydrocortisone (=free alcohol) and hydrocortisone acetate.

Typically, the topical pharmaceutical compositions according to the invention comprise the hydrocortisone component in an amount of from 0.1 up to 1.5%, especially of from 0.2 up to 1.2% and in particular of from 0.25 up to 1%, of the total composition.

The topically acceptable carriers used largely depend on the kind of topical composition involved (see below). They include e.g. aqueous phases, oily phases or emulsions but on the other hand also e.g. bandage materials, a transdermal patch environment or the typical components of a film-forming solution.

The topical compositions of the invention have valuable pharmacological properties. Especially, they are beneficial in the treatment of infections caused by dermatophytes, such as athlete's foot (tinea pedis), jock itch (tinea cruris), ringworm or onychomycosis, and also of those caused by yeasts.

It has surprisingly been found that after administration of the topical compositions of the invention patients are relieved more quickly of the symptoms accompanying superficial mycoses, such as itching, erythema, vesiculation, burning or fissures, and said superficial mycoses are in general cured more quickly.

The beneficial properties of the topical compositions of the invention can be demonstrated, for example, in the following tests.

(1) Experimental dermatophytosis model in guinea pig: It is shown [see e.g. T Itoyama et al., J Antimicrobial Chemotherapy 40 (1997) 441-444]: In said animal model, modified T. mentagrophytes-infected feet of guinea pigs are used to test terbinafine versus the combination of terbinafine with hydrocortisone. All the guinea pig feet infected with T.mentagrophytes, 10 in each group, show typical symptoms which are similar to those of naturally infected tinea pedis in humans. Aside from the fact that the course of infection is stopped very effectively by the topical compositions of the invention, there can be observed a marked reduction of inflammatory symptoms and scale formation after four days already in the group treated with the combination of terbinafine with hydrocortisone. Said effects are clearly superior to those that can be observed in the group treated with terbinafine alone.

(2) Controlled double-blind comparative study, involving ca. 480 patients with established tinea pedis who are randomized to three groups of ca. 160 each undergoing either treatment with terbinafine/hydrocortisone (1.0%/0.5%), terbinafine alone (1.0%) or placebo (vehicle). Efficacy, i.e. clinical and mycological cure, is determined at 5 days, 7 days and week 6 after the beginning of treatment.

(3) Controlled double-blind comparative study, involving ca. 640 patients with established tinea pedis who are randomized to four groups of ca. 160 each undergoing either treatment with terbinafine/hydrocortisone (1.0%/0.25%), terbinafine alone (1.0%), hydrocortisone alone (0.25% or 0.5%) or placebo (vehicle). Relief of symptoms after 1, 2 and 3 hours, 24 hours and then daily during the whole treatment period of 7 days is determined.

(4) Controlled double-blind comparative study, involving ca. 540 patients with established tinea cruris who are randomized to three groups of ca. 180 each undergoing either treatment with terbinafine/hydrocortisone acetate (1.0%/0.25%), terbinafine alone (1.0%) or placebo (vehicle). Relief of symptoms after 1, 2 and 3 hours, 24 hours and then daily during the whole treatment period of 7 days is determined.

Typically, the topically administered pharmaceutical compositions according to the invention comprise both terbinafine and hydrocortisone in pharmacologically effective amounts.

The daily dosage of the active ingredients may depend on various factors, such as sex, age, weight and individual condition of the patient. The topical pharmaceutical compositions, e.g. in the form of emulsion-gels, gels or creams, may be applied once, twice or three times daily. But also more frequent daily applications are possible. Patches, bandages and film-forming solutions may be applied, for example, once or twice daily.

The invention further relates to the use of terbinafine, or a topically acceptable salt thereof, and hydrocortisone, or a topically acceptable ester or salt thereof, (for the manufacture of a pharmaceutical composition adapted to topical administration) for the prevention or treatment of fungal infections, in particular dermatomycoses caused by dermatophytes.

Moreover, the invention relates to a method of treating fungal Infections which comprises topically administering to a mammal in need thereof a therapeutically effective amount of a mixture of terbinafine, or a topically acceptable salt thereof, and hydrocortisone, or a topically acceptable ester or salt thereof.

Pharmaceutical compositions suitable for topical administration are e.g. emulsion-gels, gels, foam gels, creams, lotions, solutions, microemulsions, ointments, fatty ointments, shampoos, pastes, foams, tinctures, film-forming solutions, nail lacquers (varnishes), bandages, patches and transdermal therapeutic systems; preferred are emulsion-gels, gels, foam gels, creams, lotions, solutions, shampoos, film-forming solutions and nail lacquers. The manufacture and composition of such topical pharmaceutical compositions are known in the art (see e.g. WO 98/00168 A1, pages 8-15 or U.S. Pat. No. 5,681,849). The concept of film-forming solutions is known in the art and e.g. disclosed in WO 98/23291 A1. The concept of nail lacquers (varnishes) is known in the art and e.g. disclosed in EP 515,312 A2.

In particular preferred is the combination of terbinafine (free base) and hydrocortisone (free alcohol or acetate) in an emulsion-gel or gel. Likewise preferred is the combination of terbinafine hydrochloride and hydrocortisone acetate in a cream (=oil-in-water emulsion).

The topical compositions of the Invention typically comprise the two active substances in dissolved or suspended form.

The following examples are intended to illustrate the invention.

### EXAMPLE 1

A gel comprising 1% terbinafine hydrochloride and 0.25% hydrocortisone (free alcohol) is manufactured as follows.

| Ingredients | Amount (g/100 g) |
|---|---|
| (A) terbinafine HCl | 1.00 |
| (B) hydrocortisone | 0.25 |
| (C) sodium pyrosulfite | 0.02 |
| (D) disodium edetate dihydrate | 0.02 |
| (E) propylene glycol | 0.70 |
| (F) hydroxypropyl cellulose (e.g. Klucel HF) | 2.00 |
| (G) Polysorbate 20 (e.g. Tween 20) | 2.00 |
| (H) ethanol 96% (v/v) | 35.00 |
| (I) water, demineralized | ad 100.0 |

(i) Dissolve A and B in a mixture of E and H.
(ii) Dissolve C, D and G in 1.
(iii) Mix (i) and (ii) at room temperature and add F.

### EXAMPLE 2

An emulsion-gel comprising 1% terbinafine (free base) and 0.25% hydrocortisone (free alcohol) is manufactured as follows.

| Ingredients | Amount (g/100 g) |
|---|---|
| (A) terbinafine | 1.0 |
| (B) hydrocortisone | 0.25 |
| (C) isopropanol | 20.0 |
| (D) propylene glycol | 5.0 |
| (E) polyhydroxyethylene cetyl stearyl ether (e.g. | 2.0 |
| Cetomacrogol 1000) | |
| (F) paraffin, liquid | 2.5 |
| (G) coco-caprylate/caprate (e.g. Cetiol LC) | 2.5 |
| (H) Carbomer 980 | 1.2 |
| (I) ammonia, concentrated aqueous solution | 1.125 |
| (J) butylhydroxytoluene | 0.02 |
| (K) water, demineralized | ad 100.0 |

(i) H is dispersed in a portion of K by means of a rotor-stator homogeniser.
(ii) A solution of B, I, J and D in C as well as the remaining K is added thereto and distributed homogeneously.
(iii) To form the fatty phase, E, G and F are melted together at 75°. A is added to the fatty phase, and then the whole fatty phase is slowly added to the previously formed gel (ii) and emulsified.

### EXAMPLE 3

An emulsion-gel comprising 1% terbinafine (free base) and 0.56% hydrocortisone acetate (corresponds to 0.5% hydrocortisone) is manufactured as follows.

| | Amount |
|---|---|
| Ingredients | (g/100 g) |
| (A) terbinafine | 1.00 |
| (B) hydrocortisone acetate | 0.56 |
| (C) Butylhydroxytoluene | 0.02 |
| (D) sodium hydroxide (pellets) | 0.10 |
| (E) benzyl alcohol | 0.50 |
| (F) Carbopol 974 P (carbomer) [= acrylic acid polymerisate] | 1.00 |
| (G) sorbitan monolaurate (e.g. Span 20) | 1.00 |
| (H) Polysorbate 20 (e.g. Tween 20) | 5.00 |
| (I) ethanol 96% (v/v) | 10.00 |
| (J) isopropyl myristate | 10.00 |
| (K) water, demineralized | ad 100.0 |

(i) A, J, C, E, G and H are mixed together with slight warming until all solid particles are dissolved.
(ii) In an appropriate vessel or processor containing a stirrer and a homogenizer about half of K is heated to 60-70° C., and B is suspended therein.
(iii) (i) is slowly added to (ii) while stirring and homogenizing until a homogeneous emulsion with appropriate droplet size is obtained. The concentrated emulsion is then cooled to room temperature.
(iv) In a separate vessel a basic carbomer gel is prepared by dispersing carbomer F in I and the second half of K and neutralizing with D.
(v) The basic emulsion (iii) is added to the basic gel and the whole is stirred at room temperature until a homogeneous emulsion gel is obtained.

### EXAMPLE 4

A cream comprising 1% terbinafine hydrochloride and 0.56% hydrocortisone acetate is manufactured as follows.

| Ingredients | Amount (g/100 g) |
|---|---|
| (A) terbinafine HCl | 1.0 |
| (B) hydrocortisone acetate | 0.56 |
| (C) isopropyl myristate | 8.0 |
| (D) Polysorbate 60 | 6.1 |
| (E) sorbitan stearate | 1.9 |
| (F) cetyl palmitate | 2.0 |
| (G) benzyl alcohol | 1.0 |
| (H) stearyl alcohol | 4.0 |
| (I) cetyl alcohol | 4.0 |
| (J) sodium hydroxide | 0.12 |
| (K) water, demineralized | ad 100.0 |

(i) C, D, E, F, G, H and I are molten together at 75° C.
(ii) A is dispersed in the major part of K and heated to 75° C.
(iii) The oily phase (i) is added to (ii) and emulsified.
(iv) The pH value is adjusted by adding J dissolved in a small portion of K, and the cream is cooled.
(v) B is suspended in a small portion of K, added to the cream and homogeneously dispersed in the latter.

1. A pharmaceutical composition adapted to topical administration comprising terbinafine, or a topically acceptable salt thereof, and hydrocortisone, or a topically acceptable ester or salt thereof, together with at least one topically acceptable carrier.

2. A composition according to claim 1, which comprises terbinafine or terbinafine hydrochloride.

3. A composition according to claim 1, which comprises hydrocortisone or hydrocortisone acetate.

4. A composition according to claim 1, wherein the terbinafine component is present in a weight percentage of from 0.1% up to 10% and the hydrocortisone component is present in a weight percentage of from 0.1% up to 1.5% of the total composition.

5. A composition according to claim 1, which is in the form of an emulsion-gel, a gel, a foam gel, a cream, a lotion or a solution, a shampoo, a film-forming solution or a nail lacquer.

6. A composition according to claim 5, which is in the form of an emulsion-gel or a gel, and which comprises terbinafine (free base) and hydrocortisone (free alcohol or acetate) as active substances.

7. A composition according to claim 5, which is in the form of a cream, and which comprises terbinafine hydrochloride and hydrocortisone acetate.

8. A method for preventing or treating a fungal infection comprising the step of topically applying a pharmaceutical composition comprising terbinafine, or a topically acceptable salt thereof, together with hydrocortisone, or a topically acceptable ester or salt thereof.

9. The method of claim 8, wherein the pharmaceutical composition prevents or treats an infection of dermatophytes.
